(19) United States
(12) Patent Application Publication
(10) Pub. No.: US 2012/0110200 A1
(43) Pub. Date: May 3, 2012

(54) Multiprotocol Adapter System and Data Conversion Method in Multiprotocol Adapter System

(76) Inventors: Eun Jin AHN, Seoul (KR); Heung Soo Park, Gyeonggi-do (KR)

(21) Appl. No.: 12/945,158

(22) Filed: Nov. 12, 2010

(30) Foreign Application Priority Data
Oct. 29, 2010 (KR) 10-2010-0107298

(51) Int. Cl. G06F 15/16

(52) U.S. Cl. 709/230

(57) ABSTRACT

Provided are a multiprotocol adapter system and a data conversion method in the multiprotocol adapter system. The multiprotocol adapter system may include a connection recognizing unit to recognize a connection with a plurality of different kinds of devices; a session managing unit to manage a connection session by sensing an input signal of the plurality of different kinds of devices connected by an input, to manage the connection with the devices; a plug and play unit to control the plurality of different kinds of devices by a unit of service provided by the plurality of different kinds of devices; a data converting unit to convert data received; a user information managing unit to manage user identity information received via radio frequency identification; and a data transmitting unit to combine the converted data and the user identity information and transmit the combined data to a terminal.

### CROSS-REFERENCE TO RELATED APPLICATION

This application claims the benefit of Korean Patent Application No. 10-2010-0107298, filed on Oct. 29, 2010, in the Korean Intellectual Property Office, the disclosure of which is incorporated herein by reference.

### BACKGROUND

1. Field of the Invention

The present invention relates to a multiprotocol adapter system and a data conversion method in the multiprotocol adapter system.

2. Description of the Related Art

Different kinds of devices may use a variety of different communication interfaces, for example, radio frequency identification (RFID), Bluetooth, universal serial bus (USB), serial communication, such as RS-232 or RS-485, and the like. Also, different kinds of devices may use different operating system platforms or different data formats.

To integratedly manage different kinds of devices using different communication interfaces and different operating system platforms or data formats, the devices need to install a plurality of operating system platforms in a management system and to purchase and install a data format conversion tool and a plurality of communication interfaces.

When a plurality of users use different kinds of devices such as medical equipment or exercise equipment in a hospital or a fitness club, it is difficult to check which device is used by which user and to obtain measurement information of a corresponding user.

Accordingly, there is a need for a system and a method for integratedly managing measurement information of a plurality of users and of different kinds of devices used by the plurality of users.

### SUMMARY

An aspect of the present invention provides a multiprotocol adapter system and a data conversion method in the multiprotocol adapter system, which may provide a communication interface with a plurality of different kinds of devices using various communication standards and may process and transmit standard/non-standard data using the communication interface, to achieve a multipurpose adaptation in a ubiquitous environment, thereby contributing to expansion of, in particular, a ubiquitous healthcare (hereinafter referred to as ‘u-healthcare’) industry by adoption of medical standards.

Another aspect of the present invention also provides a multiprotocol adapter system and a data conversion method in the multiprotocol adapter system, which may produce profits from product sales and service modeling based on business affiliation through equipment supply and service planning.

According to an aspect of the present invention, there is provided a multiprotocol adapter system including: a connection recognizing unit to recognize a connection with a plurality of different kinds of devices; a session managing unit to manage a connection session by sensing an input signal of the plurality of different kinds of devices connected by an input, to manage the connection with the devices; a plug and play unit to control the plurality of different kinds of devices by a unit of service provided by the plurality of different kinds of devices; a data converting unit to convert data received from the plurality of different kinds of devices; a user information managing unit to manage user identity information received via radio frequency identification (RFID); and a data transmitting unit to combine the converted data and the user identity information and to transmit the combined data to a terminal.

In accordance with an aspect of the present invention, the connection recognizing unit may receive, from a device management server, device information including at least one of a device identifier, a manufacturer, a model, and a device type, and may accept a connection with a corresponding device using the device information.

In accordance with another aspect of the present invention, the data converting unit may convert the received data using a data protocol provided from a manufacturer of a corresponding device, and may bypass the received data in bulk when the data protocol is not provided.

In accordance with another aspect of the present invention, the data converting unit may convert the received data by performing at least one of addition, deletion, extraction, and processing a predetermined code value on the received data using a data protocol of a corresponding device.

According to another aspect of the present invention, there is provided a data conversion method, including: recognizing a connection with a plurality of different kinds of devices; managing a connection session by sensing an input signal of the plurality of different kinds of devices connected by an input, to manage the connection with the devices; controlling the plurality of different kinds of devices by a unit of service provided by the plurality of different kinds of devices; converting data received from the plurality of different kinds of devices; managing user identity information received via radio frequency identification (RFID); and combining the converted data and the user identity information and transmitting the combined data to a terminal

### EFFECT OF THE INVENTION

The embodiments of the present invention provide a multiprotocol adapter system and a data conversion method in the multiprotocol adapter system, which may provide a communication interface with a plurality of different kinds of devices using various communication standards and may process and transmit standard/non-standard data using the communication interface, to achieve a multipurpose adaptation in a ubiquitous environment, thereby contributing to expansion of, in particular, a u-healthcare industry by adoption of medical standards.

The embodiments of the present invention provide a multiprotocol adapter system and a data conversion method in the multiprotocol adapter system, which may produce profits from product sales and service modeling based on business affiliation through equipment supply and service planning.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 schematically illustrates a multiprotocol adapter system according to an embodiment of the present invention;

FIG. 2 is a block diagram illustrating an internal structure of a multiprotocol adapter system according to an embodiment of the present invention;

FIG. 3 illustrates an example of a data conversion method according to an embodiment of the present invention;

FIG. 4 illustrates an example of a connection with devices using Bluetooth according to an embodiment of the present invention;

FIG. 5 illustrates an example of business modeling in a multiprotocol adapter system according to an embodiment of the present invention;

FIG. 6 illustrates an application example of a multiprotocol adapter system according to an embodiment of the present invention;

FIG. 7 is a flowchart illustrating a data conversion method according to an embodiment of the present invention; and

FIG. 8 is a flowchart illustrating a process for converting and transmitting data according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

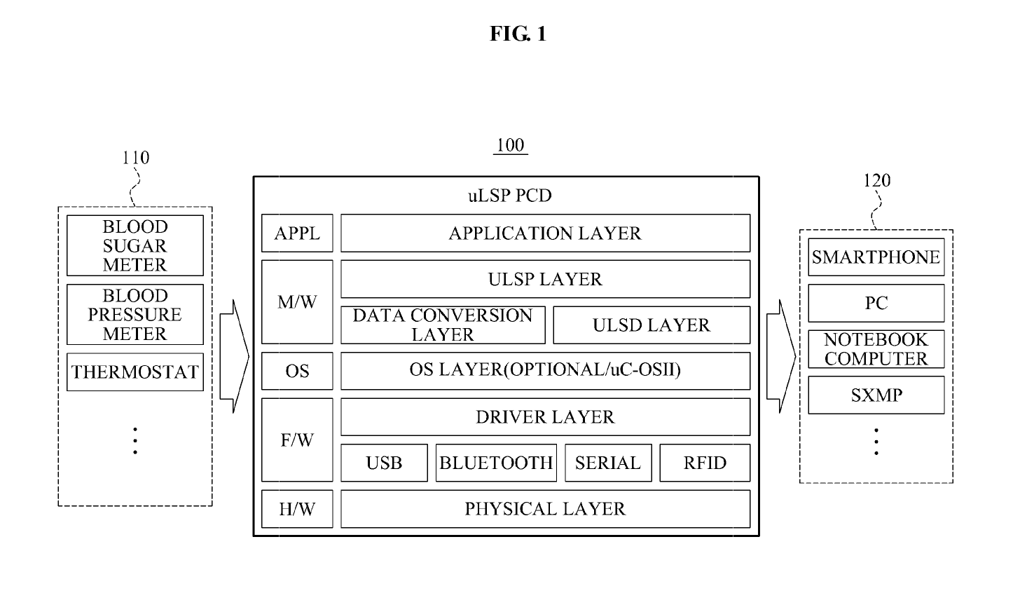

FIG. 1 schematically illustrates a multiprotocol adapter system according to an embodiment of the present invention. The multiprotocol adapter system 100 (hereinafter referred to as ‘uLSP’) according to an embodiment of the present invention may be a system that may include a wired/wireless communication processing unit based on a service plug and play (SPNP) technology and may use an embedded data processing algorithm. In other words, the uLSP 100 may receive input data of medical standards (ISO IEEE 11073 PHD) and non-standard input data of various data formats from different kinds of devices 110 using radio frequency identification (RFID), Bluetooth, universal serial bus (USB), serial communication such as Recommended Standard 232 (RS-232) or RS-485, and the like. Also, the uLSP 100 may use a data conversion algorithm for processing the input data and transmitting the input data to a terminal 120, to ensure efficiency and stability of data processing. Accordingly, the uLSP 100 may achieve a multipurpose adaptation in a ubiquitous environment, and may be expected to contribute to expansion of, in particular, a u-healthcare industry by adoption of medical standards. In this instance, the uLSP 100 may be classified into a protocol conversion device (PCD) for protocol processing and a data service device (DSD) for data processing. The uLSP 100 of FIG. 1 may include an application (APPL), a middleware (M/W), an operating system (OS), a firmware (F/W), and a hardware (H/W) in the PCD.

Specifically, the uLSP 100 may be an open source device that may provide a software development kit (SDK) for protocol conversion to a manufacturer of a measuring equipment and a manufacturer of a terminal for receiving measurement data. That is, the uLSP 100 may allow different kinds of devices using various ports to be used, and may define standards for a processor and an interface protocol for each operating system (OS) platform. In this instance, the uLSP 100 may use a simple script format. But the uLSP 100 may not store a script in processor and may not execute the script.

In this instance, the different kinds of devices 110 using various ports may include a device for measuring and providing data, for example, a blood pressure meter, a blood sugar meter, a thermostat, and the like. The terminal 120 may include a device for providing a user with data measured/transmitted by the different kinds of devices 110, for example, a smartphone, a personal computer (PC), a notebook computer, a smart cross media open platform (SXMP) as a set-top device, and the like.

Here, in the RFID technology, a tag including a microchip and an antenna may be attached to an object, and data communication between the object and a reader using radio frequency waves may be enabled. The RFID technology may be used to check the details of the object, for example, a product, and the like, to track a traveling path of the object, to manage a history of the object in real time, and the like. The RS-232C may be a standard formalized by the Electronics Industries Association (EIA), and may define an electrical factor, control handshaking, a transmission rate, a signal latency, an impedance factor, and the like, of an interface between a data terminal equipment (DTE) and a data communication equipment (DCE). However, the RS-232C may not specify a data format of and the content of data being transmitted, and may not include definition about an interface between DTEs. Most PCs may use a standard sub-set (9-pin) connector of the RS-232C for serial ports. A full standard connector may be a 25-pin D-sub connector, in which 22 pins among 25 pins may be used for communication. However, most of the pins may not be used for communication in PCs. Instead, most PCs may use 9-pin D-sub male connector. Recently, the RS-232C is being replaced by USB in PCs because the RS-232C requires a RS-232C adapter for use.

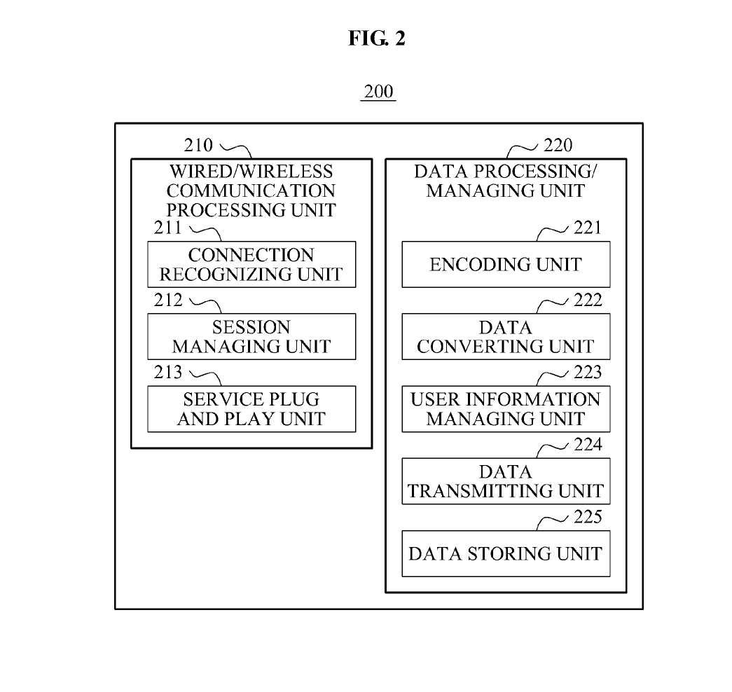

FIG. 2 is a block diagram illustrating an internal structure of a multiprotocol adapter system according to an embodiment of the present invention. As shown in FIG. 2, the multiprotocol adapter system 200 may include a wired/wireless communication processing unit 210 and a data processing/managing unit 220.

The wired/wireless communication processing unit 210 may manage a connection with a plurality of different kinds of devices. To manage a connection with a plurality of different kinds of devices, the wired/wireless communication processing unit 210 may include a connection recognizing unit 211, a session managing unit 212, and a SPNP unit 213, as shown in FIG. 2.

The connection recognizing unit 211 may recognize a connection with a plurality of different kinds of devices. For example, the connection recognizing unit 211 may include a device management module for recognizing a connection with a plurality of different kinds of devices using a serial communication such as RS-232 of an input/output type, Bluetooth, USB, and the like. The device management module may manage device information of the plurality of different kinds of devices, including a device identifier (Device ID; DID), a manufacturer, a model, and a device type, and may process acceptance/rejection of a connection with a corresponding device. The device information of a corresponding device may be received from a separate device management server (not shown). In this instance, acceptance of a connection with a corresponding device may be processed by the connection recognizing unit 211 using the device information received from the device management server, or may be processed by accepting the connection of the corresponding device by the device management server. The acceptance of a connection with a device may be processed using device management number information such as a device identifier, basic device information such as a manufacturer, a model, and a device type, management information about firmware updating, information about software of firmware, and the like. In other words, the connection recognizing unit 211 may receive, from the device management server, device information, for example, at least one of a device identifier, a manufacturer, a mode, and a device type, and may accept a connection with a corresponding device using the device information.

The session managing unit 212 may manage a connection session by sensing an input signal of a plurality of different kinds of devices connected by an input, to manage a connection with the devices. For example, the session managing unit 212 may include a response time control module, and may manage a connection session using an electrical signal between the response time control module and a connected device within a predetermined limited time, for example, about 1 minute to about 5 minutes.

The SPNP unit 213 may control a plurality of different kinds of devices by a unit of service provided by the plurality of different kinds of devices. To control a plurality of different kinds of devices, the SPNP unit 213 may register the plurality of different kinds of devices and cancel registration of the devices, and may publish a service for another device in a community using an SPNP technology. Also, the SPNP unit 213 may perform a service and may play a community-based role using the SPNP technology.

The data processing/managing unit 220 may convert data received from a plurality of different kinds of devices, and may transmit the converted data to a terminal. To convert data received from a plurality of different kinds of devices and transmit the converted data to a terminal, the data processing/managing unit 220 may include an encoding unit 221, a data converting unit 222, a user information managing unit 223, a data transmitting unit 224, and a data storing unit 225.

The encoding unit 221 may encode data received from a plurality of different kinds of devices. For example, the encoding unit 221 may encode the received data by generating a predetermined code value of a 4-bit system in a data header. In this instance, an application programming interface (API) for decoding the encoded data may be provided from a device which receives the encoded data to a manufacturer of the corresponding device. That is, an API for decoding the encoded data may be included in a corresponding device by a manufacturer of the corresponding device.

The data converting unit 222 may convert data received from a plurality of different kinds of devices. The received data may be classified into standard data and non-standard data.

When information about a manufacturer of a device which transmitted data is not obtained, data received from the corresponding device may be classified as non-standard data. In this instance, the data converting unit 222 may process and bypass the non-standard data in bulk.

When information about a manufacturer of a device which transmitted data is obtained and a data protocol is provided from the manufacturer of the corresponding device using the information about the manufacturer, data received from the corresponding device may be classified as standard data. In this instance, the data converting unit 222 may convert the corresponding standard data by performing at least one of addition, deletion, extraction, and processing of a predetermined code value on the received data. That is, the data converting unit 222 may convert the received data using a data protocol provided from the manufacturer of the corresponding device, and when the data protocol is not provided, the data converting unit 222 may bypass the received data in bulk.

The user information managing unit 223 may manage user identity information received via RFID. In this instance, the user identity information may be transmitted to the multiprotocol adapter system 200 together with data measured by different kinds of devices in, for example, a fitness club, and the like. Here, the user information managing unit 223 may manage a name, body information, for example, weight, height, and the like, a gender, an age and of a user, and the like, in addition to a user identifier (User ID; UID).

The data transmitting unit 224 may combine the converted data and the user information and may transmit the combined data to a terminal In this instance, the data transmitting unit 224 may check whether a connection with a terminal is normal, in response to a request of an agent included in the terminal, and then may transmit the combined data to the terminal

The data storing unit 225 may temporarily store the combined data of the converted data and the user information. In this instance, the data storing unit 225 may temporarily store the data in at least one case where a connection with a terminal is not normal, where the multiprotocol adapter system 200 is abnormally terminated, and where a feedback to data transmission is not provided from an agent of the terminal

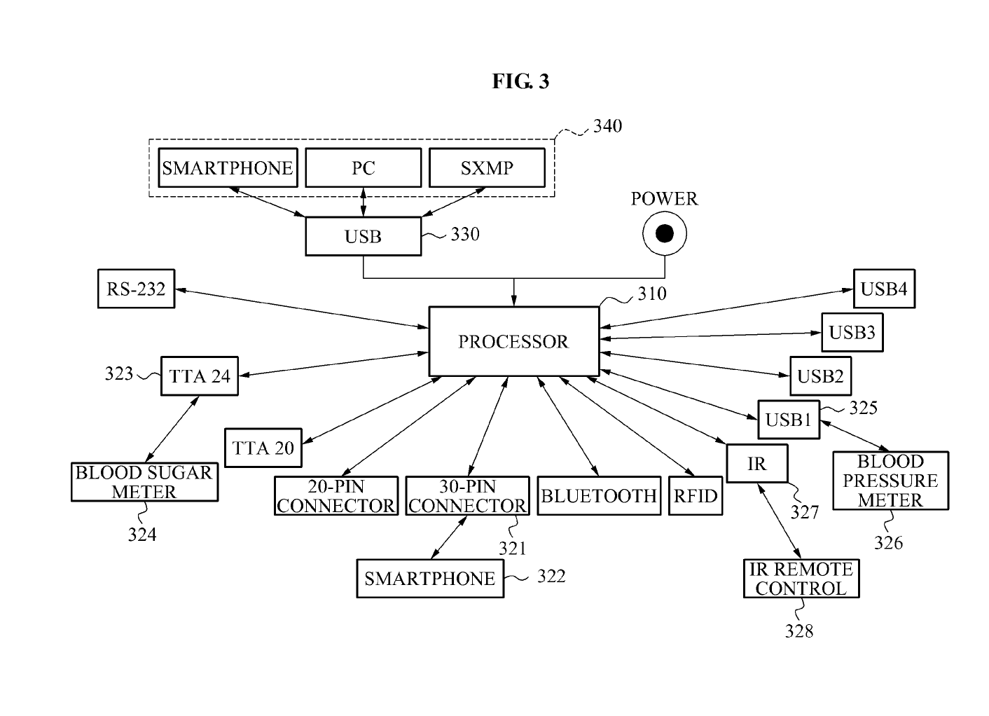

FIG. 3 illustrates an example of a data conversion method according to an embodiment of the present invention. FIG. 3 shows that a processor 310 of the multiprotocol adapter system 200 of FIG. 2 may be connected to a plurality of different kinds of devices by communication methods using various protocols. For example, the processor 310 may be connected to a smartphone 322 via a 30-pin connector 321, or may be connected to a blood sugar meter 324 via a TTA 24-pin connector 323. Also, the processor 310 may be connected to a blood pressure meter 326 via a USB1 325 among a plurality of USB ports, or may be connected to an infrared (IR) remote control 328 via an IR port 327. Here, the TTA 24-pin connector may correspond to a Telecommunications Technology Association (TTA) standard 24-pin connector, and the IR port may represent an infrared port.

When the smartphone 322 makes a request for a connection with an available port to the processor 310, the processor 310 may provide a port identifier to the smartphone 322 and may accept the connection request. In this instance, when the smartphone 322 makes a request for a device identifier to the blood sugar meter 324, the blood pressure meter 326, and the IR remote control 327 connected to the processor 310 via the TTA 24-pin connector 323, the USB1 325, and the IR port 327, respectively, each of the blood sugar meter 324, the blood pressure meter 326, and the IR remote control 327 may provide the device identifier to the smartphone 322. In this instance, a request for a device identifier may be transmitted via the processor 310, and the device identifier may be also provided via the processor 310. When the smartphone 322 includes an application or a service for both the blood sugar meter 324 and the blood pressure meter 326, the IR remote control 328 may be not used. In this instance, the smartphone 322 may make a request for release of a connection with the IR port 327 to the processor 310. An application of the smartphone 322 may perform a necessary procedure with devices such as the blood sugar meter 324, the blood pressure meter 326, and the like, until processing ends. When the processing ends, the smartphone 322 may make a request for release of a connection with all ports to the processor 310. The processing result may be provided to a user via the smartphone 322, or may be transmitted to a terminal 340 connected to the processor 310 via the USB 330 so that the processing result may be provided to another user, for example, a manager, and the like, via the terminal 340. The manager may integratedly manage blood sugar, blood pressure, and body temperature of various users, and the like. In this instance, each user may be recognized using user identity information described with reference to FIG. 2.

Also, the multiprotocol adapter system 200 may further include an interface for receiving a selection of download type from a user. For example, a variety of predetermined download types such as “A001”, “XXX0”, “XXX1”, “XXX2”, and the like, may be provided to a user through a display, for example, a liquid crystal display (LCD), and the user may select one download type using an input interface, for example, a button, and the like. In this instance, the multiprotocol adapter system 200 may execute a predetermined script depending on a download type.

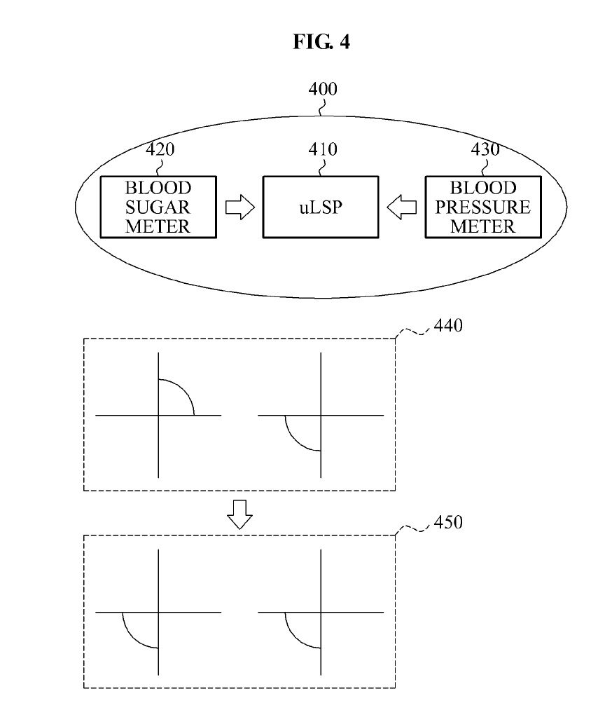

FIG. 4 illustrates an example of a connection with devices using Bluetooth according to an embodiment of the present invention. In FIG. 4, an oval 400 shows that uLSP 410, a blood sugar meter 420 and a blood pressure meter 430 form a piconet. Here, the piconet may be a small wireless network formed by connecting two or more devices to each other. An example of a connection of the uLSP 410, the blood sugar meter 420, and the blood pressure meter 430 each using Bluetooth is described with reference to FIG. 4. In this instance, the uLSP 410 may correspond to the multiprotocol adapter system 200 of FIG. 2.

Bluetooth of FIG. 4 may use 2.4 GHz bandwidth, and may have 1 Mbps symbol rate and 79 channels spaced 1 MHz apart. Bluetooth may use a frequency hopping technique that one channel is used every 625 μs. For example, a hop rate may be 1600 hops/second and each time slot may be 625 μs. A hopping sequence may be determined by a clock of a corresponding device, and in the case of a communication between two devices, a clock of a device may be synchronized with a clock of the other device. In this instance, the two devices may alternately transmit data in odd-numbered time slots and even-numbered time slot.

A connection between devices may be established through paging and inquiry procedures. That is, when one device knows a unique address BD_ADDR of the other device, a connection between the devices may be established simply through a paging procedure. When one device does not know a unique address of the other device, the device may find out the unique address of the other device through an inquiry procedure and a connection between the devices may be then established through a paging procedure.

Each device using Bluetooth may have a native clock. In this instance, because a user cannot directly control a clock of a device, a clock of a slave device may be synchronized with a system clock of a master device in a piconet. That is, dotted boxes 440 and 450 in FIG. 4 denote that a clock of the blood sugar meter 420 is synchronized with a clock of the uLSP 410 when there is a discrepancy between the clock of the blood sugar meter 420 and the clock of the uLSP 410 as a master device.

As described above, the multiprotocol adapter system 200 according to an embodiment of the present invention may cover RFID, Bluetooth, USB, and serial communication of a u-healthcare device, a u-Log device, and the like, as well as various communication/transmission standards such as standard data and non-standard data. Here, u-Log may represent device information of the u-healthcare device, for example, a model, and a set of log information, for example, a manufacturer, an equipment serial number, a connection time, a disconnection time, a usage time, and the like.

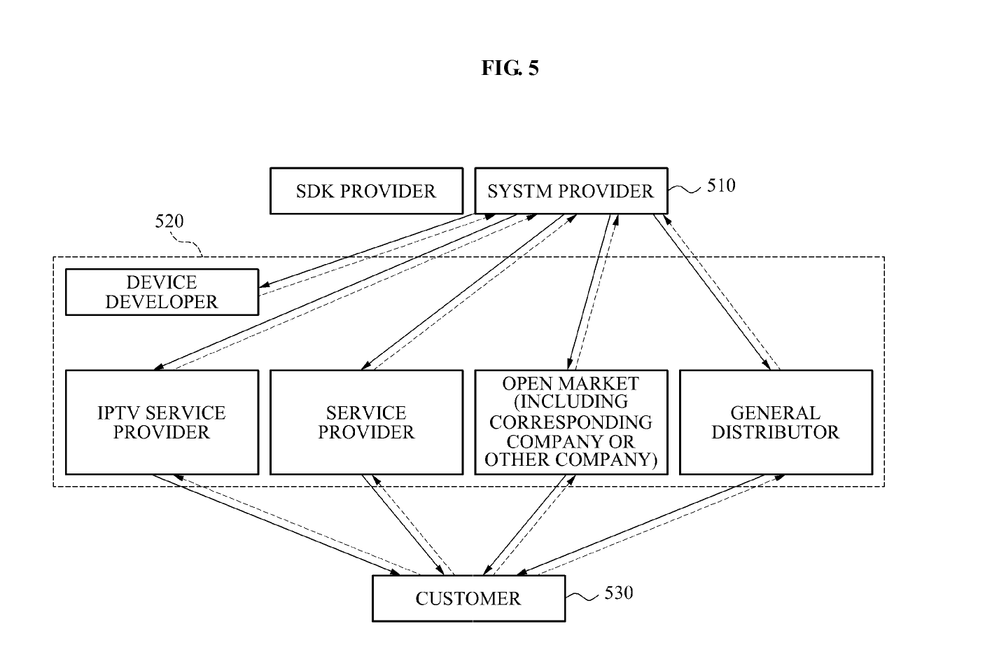

In this instance, a SDK-based device as an open source may be provided to a device developer, a service provider, an open market including a corresponding company or other company, a customer, and a general distributor, thereby producing profits from product sales and service modeling based on business affiliation through equipment supply and service planning. FIG. 5 illustrates an example of business modeling in the multiprotocol adapter system according to an embodiment of the present invention. A multiprotocol adapter system provider 510 may obtain profits by providing a multiprotocol adapter system to a businessman 520 including a device developer, an Internet protocol television (IPTV) service provider, a service provider, an open market, and a general distributor, and the businessman 520 may obtain profits by providing various kinds of services to a customer 530 using the SDK-based multiprotocol adapter system. In FIG. 5, a solid arrow may represent supply of a device or a service, and a dotted arrow may represent payment for the supplied device or service.

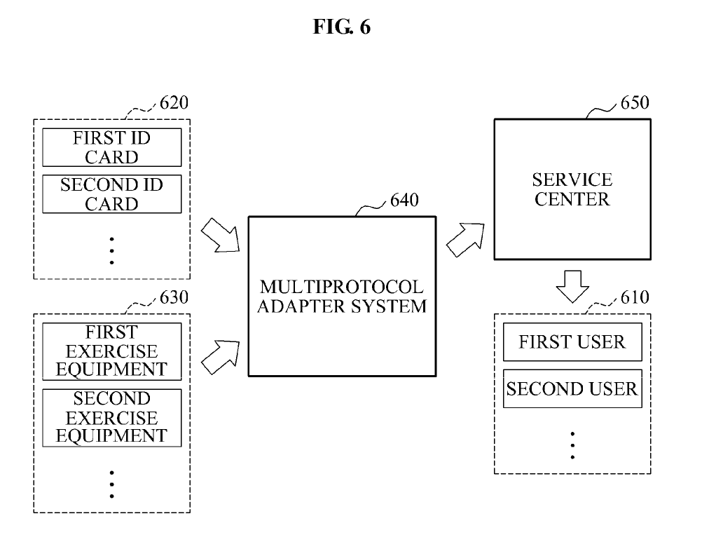

FIG. 6 illustrates an application example of the multiprotocol adapter system according to an embodiment of the present invention. In FIG. 6, ID cards 620 that may be possessed by users 610 who may receive a service, a plurality of exercise equipment 630 that may be used by the users 610, the multiprotocol adapter system 640, and a service center 650 are shown. For example, the service center 650 may be a fitness club provided with a variety of exercise equipment.

When a first user uses a second exercise equipment with a first ID card carried around the first user, user identity information of the first ID card may be provided to the multiprotocol adapter system 640 via RFID. Also, information measured by the second exercise equipment may be provided to the multiprotocol adapter system 640 via Bluetooth, IR communication, USB, or serial communication. In this instance, the multiprotocol adapter system 640 may combine the user identity information and information measured by the second exercise equipment and may transmit the combined information to a management system of the service center 650, and the service center 650 may check an exercise amount of the first user, and the like. That is, when the users 610 use the plurality of exercise equipment 630 with each ID card 620 carried around the users 610, the service center 650 may recognize standard/non-standard information received from different kinds of devices for each user using the multiprotocol adapter system 640, and may synthetically process an exercise amount of each user 610, and the like.

Although a fitness club is used as an example with reference to FIG. 6, the multiprotocol adapter system according to the present invention may be applied to any business place using different kinds of devices such as a blood pressure meter, a thermostat, a blood sugar meter, and the like, for example, a hospital, a public health center, and the like.

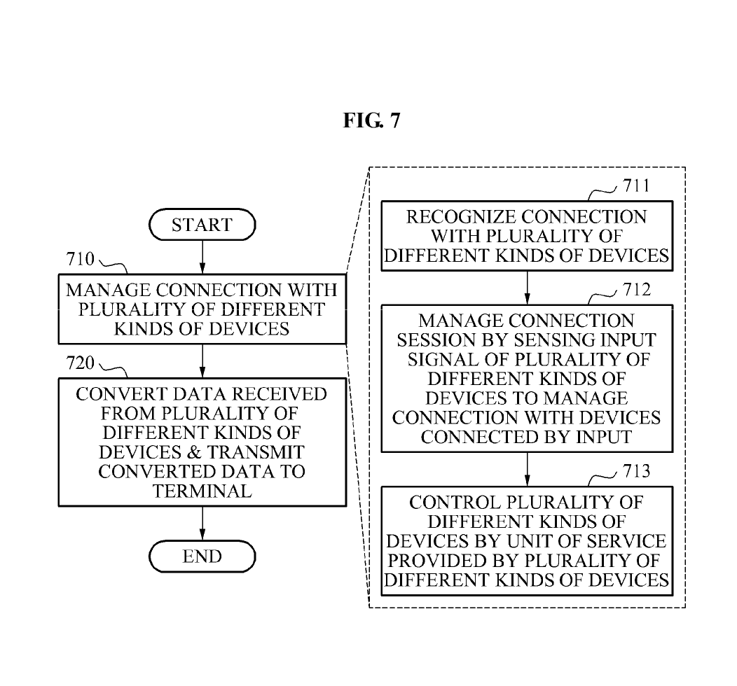

FIG. 7 is a flowchart illustrating a data conversion method according to an embodiment of the present invention. The data conversion method according to an embodiment of the present invention may be performed by the multiprotocol adapter system 200 of FIG. 2. Each operation of the data conversion method performed by the multiprotocol adapter system 200 is described with reference to FIG. 7.

In operation 710, the multiprotocol adapter system 200 may manage a connection with a plurality of different kinds of devices. To manage a connection with a plurality of different kinds of devices, the multiprotocol adapter system 200 may perform operation 710 including operations 711 to 713, as shown in FIG. 7.

In operation 711, the multiprotocol adapter system 200 may recognize a connection with a plurality of different kinds of devices. For example, the multiprotocol adapter system 200 may include a device management module for recognizing a connection with a plurality of different kinds of devices using Bluetooth, USB, serial communication such as RS-232 of an input/output type, and the like. In this instance, the device management module may manage device information of a plurality of different kinds of devices, for example, a device identifier (device ID; DID), a manufacturer, a model, and a device type, and may process acceptance/rejection of a connection with a corresponding device using the device information. Here, device information of a corresponding device may be received from a separate device management server (not shown). In this instance, acceptance of a connection with a corresponding device may be processed by the connection recognizing unit 211 using device information received from the device management server or may be processed by accepting the corresponding device by the device management server. The acceptance of a connection with a device may be processed using device management number information such as a device identifier, basic device information such as a manufacturer, a model, and a device type, management information about firmware updating, information about software of firmware, and the like. In other words, the multiprotocol adapter system 200 may receive, from the device management server, device information, for example, at least one of a device identifier, a manufacturer, a model, and a device type, and may accept a connection with a corresponding device using the device information.

In operation 712, the multiprotocol adapter system 200 may manage a connection session by sensing an input signal of a plurality of different kinds of devices connected by an input, to manage a connection with the devices. For example, the multiprotocol adapter system 200 may include a response time control module, and may manage a connection session using an electrical signal between the response time control module and a connected device within a predetermined limited time, for example, about 1 minute to about 5 minutes.

In operation 713, the multiprotocol adapter system 200 may control a plurality of different kinds of devices by a unit of a service provided by the plurality of different kinds of devices. To control a plurality of different kinds of devices, the multiprotocol adapter system 200 may register the plurality of different kinds of devices and cancel registration of the devices, and may publish a service for another device in a community using an SPNP technology. Also, the multiprotocol adapter system 200 may perform a service and may play a community-based role using the SPNP technology.

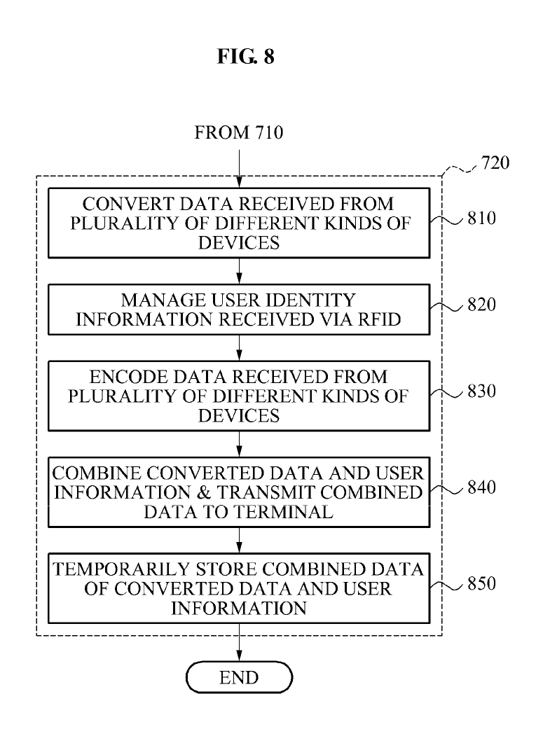

In operation 720, the multiprotocol adapter system 200 may convert data received from the plurality of different kinds of devices and transmit the converted data to a terminal. Operation 720 is described in detail with reference to FIG. 8.

FIG. 8 is a flowchart illustrating a process for converting and transmitting data according to an embodiment of the present invention. In this instance, operations 810 to 850 of FIG. 8 may be included in operation 720 of FIG. 7.

In operation 810, the multiprotocol adapter system 200 may convert data received from the plurality of different kinds of devices. In this instance, the received data may be classified into standard data and non-standard data.

When information about a manufacturer of a device which transmitted data is not obtained, data received from the corresponding device may be classified as non-standard data. In this instance, the multiprotocol adapter system 200 may process and bypass the non-standard data in bulk.

When information about a manufacturer of a device which transmitted data is obtained and a data protocol is provided from the manufacturer of the corresponding device using the information about the manufacturer, data received from the corresponding device may be classified as standard data. In this instance, the multiprotocol adapter system 200 may convert the corresponding standard data by performing at least one of addition, deletion, extraction, and processing of a predetermined code value on the received data.

In operation 820, the multiprotocol adapter system 200 may manage user identity information received via RFID. In this instance, the user identity information may be transmitted to the multiprotocol adapter system 200 together with data measured by different kinds of devices in, for example, a fitness club, and the like. Here, the user information managing unit 223 may manage a name, body information, for example, weight, height, and the like, a gender, an age of a user, and the like, in addition to a user identifier (User ID; UID).

In operation 830, the multiprotocol adapter system 200 may encode the data received from the plurality of different kinds of devices. For example, the multiprotocol adapter system 200 may encode the received data by generating a predetermined code value of 4-bit system in a data header. In this instance, an application programming interface (API) for decoding the encoded data may be provided from a device which receives the encoded data to a manufacturer of the corresponding device. That is, an API for decoding the encoded data may be included in a corresponding device by a manufacturer of the corresponding device.

In operation 840, the multiprotocol adapter system 200 may combine the converted data and the user information and may transmit the combined data to a terminal. In this instance, the multiprotocol adapter system 200 may check whether a connection with a terminal is normal, in response to a request of an agent included in the terminal, and then may transmit the combine data to the corresponding terminal

In operation 850, the multiprotocol adapter system 200 may temporarily store the combined data of the converted data and the user information. In this instance, the data storing unit 225 may temporarily store the data in at least one case where a connection with a terminal is abnormal, where the multiprotocol adapter system 200 is abnormally terminated, and where a feedback to data transmission is not provided from an agent of the terminal

Accordingly, the present invention may process and transmit standard/non-standard data via a communication interface with a plurality of different kinds of devices using various communication standards, to achieve a multipurpose adaptation in a ubiquitous environment, thereby contributing to expansion of, in particular, a u-healthcare industry by adoption of medical standards. Also, the present invention may produce profits from product sales and service modeling based on business affiliation through equipment supply and service planning.

The above-described exemplary embodiments of the present invention may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described exemplary embodiments of the present invention, or vice versa.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A multiprotocol adapter system, comprising:
a connection recognizing unit to recognize a connection with a plurality of different kinds of devices;
a session managing unit to manage a connection session by sensing an input signal of the plurality of different kinds of devices connected by an input, to manage the connection with the devices;
a plug and play unit to control the plurality of different kinds of devices by a unit of service provided by the plurality of different kinds of devices;
a data converting unit to convert data received from the plurality of different kinds of devices;
a user information managing unit to manage user identity information received via radio frequency identification (RFID); and
a data transmitting unit to combine the converted data and the user identity information and to transmit the combined data to a terminal

2. The multiprotocol adapter system of claim 1, wherein the connection recognizing unit receives, from a device management server, device information including at least one of a device identifier, a manufacturer, a model, and a device type, and accepts a connection with a corresponding device using the device information.

3. The multiprotocol adapter system of claim 1, wherein the data converting unit converts the received data using a data protocol provided from a manufacturer of a corresponding device, and bypasses the received data in bulk when the data protocol is not provided.

4. The multiprotocol adapter system of claim 1, wherein the data converting unit converts the received data by performing at least one of addition, deletion, extraction, and processing a predetermined code value on the received data using a data protocol of a corresponding device.

5. A data conversion method, comprising:
recognizing a connection with a plurality of different kinds of devices;
managing a connection session by sensing an input signal of the plurality of different kinds of devices connected by an input, to manage the connection with the devices;
controlling the plurality of different kinds of devices by a unit of service provided by the plurality of different kinds of devices;
converting data received from the plurality of different kinds of devices;
managing user identity information received via radio frequency identification (RFID); and
combining the converted data and the user identity information and transmitting the combined data to a terminal.
